(19) United States
(12) Patent Application Publication
(10) Pub. No.: US 2006/0211766 A1
(43) Pub. Date: Sep. 21, 2006

(54) Gelled immunomodulating topical compositions and a method of treating warts and other human papilloma virus skin infections

(76) Inventors: Leonard L. Kaplan, East Brunswick, NJ (US); William R. Levis, New York, NY (US)

Correspondence Address: LEONARD L. KAPLAN, ONE MINUTE MAN CT., EAST BRUNSWICK, NJ, 08816, US

(21) Appl. No.: 11/228,569

(22) Filed: Sep. 16, 2005

Related U.S. Application Data
(60) Provisional application No. 60/614,577, filed on Sep. 30, 2004.

(51) Int. Cl. A61K 31/215; A61K 31/135; A61K 31/12; A61K 31/05

(52) U.S. Cl. 514/529; 514/690; 514/646; 514/731

(57) ABSTRACT

Topical drug compositions of this invention contain delayed type contact sensitizing haptens in a unique non-flowable, non-toxic, non-volatile, anhydrous gel composition to achieve retained site application on warts and other human papilloma virus (HPV) skin infections. The preferred gelled compositions contain, but are not limited to, the sensitizing haptens, squaric acid dibutylester and diphenylcyclopropenone in optimized blends of Polysorbate 80, Isopropyl myristate uniquely gelled with Polyoxyl 40 stearate to form a penetrant of keratinized epitheliiuim of warts for direct application wherein virucidal pharlacologic action is induced by Th-1 cell mediated immune responses with resultant releases of CD4 helper T cells, CD8 killer T cells and cytokines to attack the human papilloma viruses. The commonly used vehicles with these contact sensitizers are acetone, petrolatum, or water containing emulsion creams which do not have the capacity to penetrate the keratinized wart surfaces and are therefore minimally effective in treating warts.

### RELATED US PATENTS CITED

U.S. Pat. No. 4,985,464 Happle & Hauser, “Drug Compositions for Local Treatment of Alopecia Areata,” (1991)

U.S. Pat. No. 6,455,586, Kaplan & Levis, “Topical Immunomodulating Compositions for Treatment of AIDS, Hepatitis, other Infectious Diseases and Cancer,” (2002)

U.S. Pat. No. 6,761,900, Shudo & Mori, “Topical Patch Preparation containing a Delayed Type Hypersensitivity Inducer and Methods for Using the Same,” (2004)

### RELATED US APPLICATION

Provisional Patent Application No. 60/614,577 filed Sep. 30, 2004 “Gelled Immunomodulating Topical Compositions for Treatment of Warts and other Human Papilloma Viral Infections.”

### SUMMARY OF THE INVENTION

This invention provides unique non-flowable gel compositions as a method for direct and retained topical application of delayed type contact sensitizer haptens in an anhydrous solubilized drug delivery system to penetrate the keratinized epithelium of human papilloma virus infections on the skin to reach the antigen presenting cells in the dermis. This results in a Th-1 cell mediated immune response with release of antiviral cytokines, CD4 helper T cells and CD8 killer T cells that attack the virus.

The preferred active delayed type contact sensitizer embodiments of this invention are the chemical haptens squaric acid dibutylester and diphenylcyclopropenone. Other haptens covered by the invention in oxazolone, dinitrochlorbenzene, dinitrofluorobenzene, urishiol and paraphenylenediamine. The preferred gel drug delivery system embodiments of this invention consist of polysorbate 80, Isopropyl myristate and palmitate and polyoxyl 40 stearate that constitute an anhydrous, non-volatile, non-toxic, non-flowable, keratinized epithelium penetrating vehicle that is skin absorbable to reach the dermal dendritic cells that are the primaryantigen presenting cells of the human cellular immune system.

This invention provides unique therapeutic advantages of acetone based solutions, petrolatum based vehicles and aqueous creams that are not reliably absorbed through the keratinized epithelium. Acetone is volatile, toxic and irritating and unreliable in delivering the active ingredients. Petrolatum is greasy and does not absorb through the skin. The entire contents of the related immunomodulating compositions in U.S. Pat. No. 6,455,586 are cited by reference and fully incorporated herein.

### FIELD OF INVENTION

This invention is in the field of topical medical compositions of Immunomodulating haptens of the class of contact sensitizers to induce delayed type Th-1 cellular immune hypersensitization reactions to cure human papilloma virus (HPV) infections, particlarly common warts, plantar warts and genital warts delivered in a novel topical non-aqueous, non-volatile, non-irritating, non-flowable gelled form applied directly to warts lesions and so retained on the lesion due non-flowability of controlled viscosity gel without the need for a cumbersome skin adhesive patch preparation containing water soluble polymer gels normally required to deliver a delayed type hypersensitivity agent.

### BACKGROUND OF THE INVENTION

Contact sensitizer haptens from the group containing such as squaric acid dibutylester, diphenylcyclopropenone, dinitrochlorobenzene, dinitrofluorobenzene, oxazolone, paraphenylenediamine and urushiol represent a medical field of investigation known as immune response modulators useful for the treatment of human papilloma viral infections. Topical imiquimod is included in the drug class of immune response modifiers which induce antiviral cytokine activity via the release of interferon. Cell mediated immune function rather than humoral immune function is recognized medically as the basis for this anti-viral activity based on increases in CD4 helper and CD8 killer T cells. Biologically, this results from Th-1 cell mediated immune responses with release of cytokines to impart human papilloma virucidal activity. Warts as human papilloma infections can occur in various forms, i.e., common warts (verruca vulgaris), plantar warts (verruca plantaris) & genital warts including venereal and inguinal warts. These are prevalent among all racial groups.

In addition, the human papilloma viruses may play a role in development of basal cell carcinoma and melanoma as well as cervical cancer in women with genital warts infections.

Current therapies as warts treatments are divided into three groups: cellular destruct chemical agents, cryotherapy and immunomodulators. Cellular destructive therapies include chemical agents such as podophyllin, trichloroacetic acid, 5-fluorouracil, bleomycin, retinoids, glutaraldehyde, formaldehyde, salicylic acid and cantharidin. With cryotherapy, the wart is exposed to liquid nitrogen for several treatments spread over three to four weeks required to destruct the warts cellular structure. These treatments results in varying success depending on the nature and severity of the wart lesions.

Contact sensitizer immunomodulating therapy has been used as a topical treatment for multiple, recalcitrant viral warts in petrolatulml. Diphenylcyclopropenone and squaric acid dibutyl ester represent the main embodiments of this uniquely gelled immunotherapeutic invention formulated in a non-irritating, non-volatile epidermal penetrating anhydrous gelled vehicle composition containing polysorbate 80 surfactant, isopropyl myristate emollient and polyoxyl 40 stearate as the gelling agent. The commonly used vehicles for topical contact sensitizer alopecia areata treatments consist of (a) acetone to enhance solution of the contact sensitizer and (b) petrolatum as a viscous non-flowable application for wart sites. These formulations are questionably effective due to the shortcomings of (a) skin irritation, poor stability, solvent volatility and unreliable skin penetration with acetone, and (b) poor bioavailability and unreliable penetration to the warts cellular tissue level with petrolatum. Accordingly, there is a need for reliable, stable, non-irritating and HPV lesion penetrating bioavailable compositions for retained topical application to common, plantar and genital warts.

### DETAILED DESCRIPTION OF THE INVENTION

The immunomodulating gel compositions of this invention treat human papilloma virus infections embodied by common warts, plantar warts and genital warts wherein the patient's immune system is induced by the immune modulating contact sensitizer known chemically as haptens to release CD4 helper T cells, CD8 killer T cells and cytokines that attack the human papilloma virus causing the warts. The active ingredients in these compositions are those that are haptens classified pharmacologically as delayed type contact sensitizers. These include diphenylcyclopropenone, squaric acid dibutylester, dinitrochlorobenzene, dinitrofluorobenzene, oxazoline, paraphenylenediamine, urishiol and the like that are generally recognized as Th-1 cell mediated immune inducers. Width penetration of the keratinized stratum corneum of warts lesions to the cellular layer, these compounds have been shown to raise CD4, CD8 T cells and cytokines to exercise virucidal activity and reduce warts lesions. The commonly used vehicle for these topically applied contact sensitizers have been solutions in acetone ass the vehicle. Acetone is a volatile and highly inflammable organic liquid with an LD/50 of 10.7 mg per kilogram in rodents and is classifed as a toxic substance. Repeated human use causes epidermal erythema, skin dryness, headache, bronchial irritation, CNS effects including fatigue and excitement and with chronic use narcosis. In addition, due due its high vapor pressure and volatility, the contact sensitizer-acetone solutions when applied to the skin can result in variable, inconsistent and unreliable levels of contact sensitizer absorption through the skin. The other commonly used carrier for contact sensitizers is petrolatum, which a highly viscous, non-skin absorbable unctious material that does not reliably release the contact sensitizers contained therein, and does not permit reliable bioavailability when applied to warts lesions. In order for the topically applied contact sensitizers to be optimally safe and effective in reducing warts caused by human papilloma viruses, we found unexpectedly that the topical compositions of this invention consisting of medically accepted delayed type contact sensitizers solubilized in drug delivery vehicles of non-volatile, anydrous, controlled viscosity non-flowable gels consisting of non-ionic surfactants and cosmetically acceptable emollient esters of fatty acids embodied by isopropyl myristate and palmitate gelled with polyoxy 40 stearate result in effective absorption through the keratinized epidermis of viral warts to release CD4 and CD8 T cells and cytokines that effectively kill the human papilloma virus that causes the warts formations on the skin.

The drug compositions of this invention accordingly may contain non-ionic surfactants of the following classes:

Poyoxyethylene (POE) sorbitan fatty acid esters identified generically as POE 20 sorbitan monolaurate, POE 20 sorbitan monopalmiate, POE 20 sorbitan monostearate, POE 20 sorbitan monooleate, POE 20 sorbitan monotriooleate and the like that are oily liquids with low vapor pressure properties and therefore non-volatile and non-irritating to the skin and have the property of emulsifying and dissolving immiscible combinations of the active contact sensitizers and the emollient co-solvents embodied by the following alcoholic esters of myristic and palmitic fatty acids:

Isopropyl myristate consisting of esters of isopropyl alcohol and saturated high molecular weight fatty acids, principally myristic acid; and Isopropyl palmitate consisting of ester of isopropyl alcohol and saturated high molecular weight fatty acids, principally palmitic acid, and other like alcohol esters of saturated high molecular weight fatty acids that are mobile oily liquds at room temperature.

In order to change the above listed co-solvent solutions containing the contact sensitizers to a gelled non-flowable physical form suitable for topical application to skin warts, it was unexpectedly found that controlled addition of polyoxyl 40 stearate has the unique properties of forming a non-flowable application to warts while mainitaining the absorption properties through the keratinized epidermis of the warts. Polyoxyl 40 stearate is contained in the class of polyethylene glycol esters of fatty acids wherein a wide range of properties can be achieved by controlling the hydrophobic fatty acid segment of the polymer. Polyoxyl 40 stearate is a mixture of mono and distearate esters of polyoxyethylene with an average number of oxy ethylene units of 40. It was unexpectedly found that addition of polyoxy 40 stearate to the surfactants and alcoholic ester emollients listed above results in a unique anydrous gel composition uniquely suited for applied delivery of the contained contact sensitizer active ingredient to warts being treated.

The drug compositions of this invention are best administered directly to the warts to permit localized absorption through the warts' keratinized epithelium and may be covered by an occlusive or semi-occlusive dressing if desired.

Contact sensitizers contained in optimized gelled vehicles are described in the following examples:

| | | | |
|---|---|---|---|
| (1) | Diphenylcyclopropenone | 0.001% | w/w |
| | POE (20) sorbitan monooleate | 45.000% | w/w |
| | Isopropyl myristate | 45.000% | w/w |
| | Polyoxyl 40 stearate | 9.999% | w/w |
| (2) | Diphenylcyclopropenone | 4.000% 5 | w/w |
| | POE (20) sorbitan monoleate | 44.000% | w/w |
| | Isopropyl myristate | 44.000% | w/w |
| | Polyoxyl 40 stearate | 8.000% | w/w |
| (3) | Squaric Acid Dibutylester | 0.010% | w/w |
| | POE (20) sorbitan monolaurate | 45.000% | w/w |
| | Isopropyl palmitate | 45.000% | w/w |
| | Polyoxyl 40 Stearate | 9.990% | w/w |
| (4) | Squaric Acid Dibutylester | 4.000% | w/w |
| | POE (20 sorbitan monolaurate | 44.000% | w/w |
| | Isopropyl palmitate | 44.000% | w/w |
| | Polyoxyl 40 Stearate | 8.000% | w/w |
| (5) | Dinitrochlorobenzene | 2.000% | w/w |
| | POE (20) monopalmitate | 46.000% | w/w |
| | Isopropyl myristate | 46.000% | w/w |
| | Polyoxyl 40 Stearate | 10.000% | w/w |

Apply 0.1 gram to 1.0 gram quantities of the gel directly on warts surfaces. Cover with an occlusive or non-occlusive dressing to prevent transference of the gel to non-warts areas on the skin.

These composition examples are cited to demonstrate, but not to limit various concentrations of active contact sensitizer gels applied to warts surfaces. Other examples of contact sensitizers from the group to be applied in the unique non-volatile, non-irritating, non-flowable, skin absorbable vehicle compositions are dinitrofluorobenzene, oxazoline, phenylenediamine, urishiol and others in the pharmacologic class of delayed type hypersensitization contact sensitizers.

As an example of the efficacy of the described contact sensitizer gel compositions, three patients were treated over an 8 week period with the following squaric acid dibutyl ester gel compositions:

Treatment regimens: Initial sensitization with 2% squaric acid dibutylester gel at primary wart wite. Cover with semi-occlusive pad for 48 hours and removed. Treat weekly with 0.2% squaric acid dibutylester for 10 weeks.

Warts Clearance Global Assessment Scoring: 0—complete warts clearance; 1—50% or greater warts clearance; 2—<50% warts clearance; 3—warts present, no clearance of warts.

TABLE 1
18 year old caucasian male with 3 wart lesions on left foot. 7 weekly
treatments after initial sensitization.

Warts Clearance Results: 3rd week - 2 score partial <50% warts clearance
Warts Clearance Results: 4th week - 1 score >50% warts clearance
Warts Clearance Results: 7th week - 1 score >50% warts clearance

TABLE 2
21 year old caucasian male with wart on right thumb. 7 weekly
treatments after initial sensitization.

Warts Clearance Results: 2nd week - 2 score partial <50% warts clearance
Warts Clearance Results: 3rd week - 1 score >50% warts clearance
Warts Clearance Results: 7th week - 1 score >50% warts clearance

TABLE 3
26 year old hispanic male with multiple warts (30+) in groin area.
8 weekly treatments after initial senitization

Warts Clearance Results: 2nd week - 2 score partial <50% warts clearance
Warts Clearance Results: 4th week - 1 score >50% warts clearance
Warts Clearance Results: 8th week - 0 score complete warts clearance

### CONCLUSIONS

1. Warts patients in this proof of concept study were all sensitized with 2% squaric acid dibutylester gel topical applications to the warts sites.
2. Weekly topical applications of 0.2% squaric acid dibutylester gel for 7-8 weeks
3. All patients responded favorably to the weekly topical applications.
4. Patient #3 with 30+ groinal warts showed complete clearance after 8 weeks
5. Patients # 2 and # 1 showed >50% clearance after 7 weeks.
6. None of the patients complained of side effects or adverse events.
7. The results of this proof of concept contact sensitizer immunotherapy study with 2% and 0.2% squaric acid dibutylester gel of this invention demonstrated that warts in different parts of the body were safely and effectively treated with the squaric acid dibutylester gels over a 7-8 week period.

1. A method of topically treating human papilloma virus verruca diseases including common warts, plantar warts, inguinal warts and venereal warts comprised of anhydrous, non-volatile, non-irritating gels wherein the gels are non-flowable to be retained on the warts surface and wherein the the gels are uniquely formulated with surfactants and emollients to penetrate the keratinized epithelium of warts surfaces to provide therapeutically effective anti-viral activity.

2. The compositions of claim 1 consisting of delayed type contact sensitizer hapten selected from the group consisting of squaric acid dibutylester, diphenylcyclopropenone, dinitrochlorobenzene, dinitrofluorobenzene, exanolone, paraphenylenediamine and urishiol, and a first co-solvent selected from the group consisting of polyoxyethylene 20 monoleate, palmitate and stearate, and a second co-solvent consisting of isopropyl myristate orisopropyl palmitate, and a gelling agent polyoxyl 40 stearate.

3. A composition of claim 2 wherein the preferred delayed type contact sensitizer hapten embodiment is squaric acid dibutylester comprised from about 0.001% to 4.000% by weight.

4. A composition of claim 2 wherein the preferred delayed type contact sensitizer hapten embodiment is diphenylcyclopropenone comprised from about 0.001% to 4.000% by weight.

5. A method of treating human papilloma virus associated skin diseases including actinic keratosis pre-cancer lesions comprised of a topically applied non-flowable anhydrous, non-volatile non-toxic gel composition, wherein the composition consists of a delayed typed contact sensitizer hapten selected from the group in claim 2, and gel carrier comprised of a polyoxyl 40 fatty acid ester and a first co-solvent and a second co-solvent selected from the groups in claim 2.

6. The method of claim 5. wherein the preferred delayed type contact sensitizer hapten is squaric acid dibutyester comprised from about 0.001% to 4.000% by weight.

7. The method of claim 5. wherein the preferred delayed type contact sensitizer hapten is diphenylcyclopropenone comprised from about 0.001% to 4.000% by weight.

8. The method of claim 1. wherein said gel compositions are first applied to the warts skin lesion site and secondly covered by an absorbent pad under an occlusive or semi-occlusive backing for localized retention and absorption through the keratinized epithelium of the warts.

9. The method of claim 5. wherein said gel compositions are first applied to the human papilloma virus skin lesion site and secondly covered by an absorbent pad under and occlusive or semi-occlusive backing for localized retention and absorption through the keratinized epithelium of the lesion/
